(19) United States
(12) Patent Application Publication
(10) Pub. No.: US 2002/0068046 A1
(43) Pub. Date: Jun. 6, 2002

(54) Use of stem cells derived from dermal skin

(76) Inventors: Jianwu Dai, Boston, MA (US); Eugene Bell, Boston, MA (US)

Correspondence Address: Ellen M. Leonnig, TEI Biosciences, Inc., 7 Elkins Street, Boston, MA, 02127, US

(21) Appl. No.: 09/901,786

(22) Filed: Jul. 9, 2001

Related U.S. Application Data
(60) Provisional application No. 60/256,614, filed on Dec. 18, 2000.
(60) Provisional application No. 60/256,593, filed on May 29, 2001.
(60) Provisional application No. 60/251,125, filed on Dec. 4, 2000.

(51) Int. Cl. A61K 45/00; C12N 5/08

(52) U.S. Cl. 424/93.7; 435/366; 424/423

(57) ABSTRACT

The applications of this invention, the use of stem cells derived from the dermis, include but are not limited to cell differentiation, histiogenesis, organogenesis, cell therapy, tissue engineering, and tissue and organ regeneration.

### CROSS-REFERENCE TO RELATED APPLICATIONS

This application is related to co-pending U.S. application Ser. Nos. 60/256,614, filed Dec. 18, 2000, 60/256,593, filed Dec. 18, 2000 and 60/251,125, filed Dec. 4, 2000 (from which it claims priority), and a U.S. application filed concurrently, entitled “Generation and Use of Signal-plexes to Develop Specific Cell Types, Tissues and/or Organs,” the entire contents of which are incorporated herein by reference.

### BACKGROUND OF THE INVENTION

1. Field of the Invention

This invention relates to the field of tissue engineering, particularly to stem cell technology. A critical quest for those interested in the use of stem cells in biotechnology and regenerative medicine is the search for cells which because of their position and history in the developing embryo or fetus, retain some degree of multipotency (the capacity to become one of a number of different cell types). These cells may provide an important resource, as valuable as the pluripotent cells of the innercell mass of the blastula, for rebuilding the human body in need of new parts.

Stem cells are cells from the embryo, fetus or adult which have the capacity to become different cell types when presented with specific signaling complexes that provide the directions to do so. Signals which cause cells to differentiate, and participate in tissue and organ development come from the cells themselves, from neighboring cells, and also from distant cells, such as endocrine cells. In the course of embryonic and fetal development, the signals that cells generate and receive depend on their position in the developing organism. Small differences among cells can predispose them to become part of a particular developmental lineage. The differences can arise very early, as the egg cytoplasm divides giving each blastomere a fraction of the molecules present in the egg, which from the outset are asymmetrically distributed.

As cell divisions in mammals progress, early in development a blastula or hollow ball of cells forms and at one pole of the hollow ball, a mass of cells, called the inner cell mass, appears as a result of a string or cell divisions creating a population of about 128 to 250 cells called pluripotent stem cells capable of making all parts of the developing organism except the extra-embryonic membranes. Under certain conditions in vitro these cells can be kept in cycle indefinitely. At any time however, it is possible with the appropriate manipulations, to activate the developmental capacity with which these cells are endowed, by allowing the cells to assemble into aggregates. These aggregates are called embryoid bodies (EBs), within which the cells engage in random exchange of signals leading to the disorganized differentiation of a great variety of cells, tissues and structures, rather than to the highly organized embryo that emerges in the course of normal embryonic development.

Early in human development the three germ layers, ectoderm, mesoderm and endoderm are formed as a result of cell movements and interactions, each giving rise to a predictable lineage of tissue and organ derivatives. The morphogenetic rearrangement of cells establishes subpopulations, neighborhoods and neighbors that interact and specialize as molecular signals are secreted and inducing adjacent cells as well as the cells which secrete them to undergo divisions, engage in morphogenesis and differentiate into tissues and organs.

2. Description of the Related Art

Two general classes of stem cells have been identified, one called pluripotent embryonic stem cells [Thompson J A et al, Science 282, 1145-47 (1998); Gearhart J, Science 282, 1061-62 (1998)] and the other adult stem cells harbored in the bone marrow and known about for some years. In addition to the blood forming and immune cell populations, various cell types of the skeletal system such as bone, tendon, cartilage and ligament cells, endothelial precursor cells, cardiomyocytes and nerve cell progenitor cells have been identified.

### BRIEF DESCRIPTION OF THE INVENTION

Cells from the human fetus, particularly dermal fibroblasts from skin taken from fetuses between the ages of 8 and 24 weeks, have been shown to differentiate into tissue cells having the features of, e.g., endocrine pancreas, exocrine pancreas, liver, cartilage, bone muscle and kidney, under the influence of signaling complexes designed to induce specifically the foregoing phenotypes. Signaling complexes are described in a concurrently filed U.S. Patent application referred to herein.

This invention has significant advantages for cell therapy, tissue engineering, or other related purposes. It is technically much simpler and more economical than other procedures. In addition, it may also produce genetically matched cells for cell therapy and tissue engineering.

### DETAILED DESCRIPTION OF THE INVENTION

It is believed that mesenchymal cells such as dermal fibroblasts, or fibroblasts from any other fetal, neonatal or adult source of any age may harbor stem cell populations capable of differentiating or transdifferentiating into multiple cell phenotypes if induced to do so with the appropriate developmental signals. The principal known source of stem cells in the adult are the mesenchymal stem cells or fibroblasts of the bone marrow, shown to be capable of differentiating into cells of the skeletal system (e.g., bone, cartilage, tendon, endothelial, cardiac and neural cells). It is highly probable that specific signaling complexes, e.g., those produced and used by the methods described in a concurrently filed application referred to herein, are capable of inducing a much broader range of phenotypes in the fibroblast population found in the bone marrow and predictably, from fibroblastic reservoirs which may come from other tissues (e.g., lung, connective and muscle tissue). This discovery provides an opportunity to produce specific cells in large quantity for cell therapy and tissue engineering.

A property of the stem cells of the dermal tissue which distinguishes them is the induced expression of the stem cell marker CD34 expressed after the stem cells are cultured and passaged in DMEM with an extract of mES (mouse embryonic stem cells). mES cells are spun into a pellet and resuspended in PBS and sonicated at 4° C. for between 5 and 10 seconds about 10 times with 10.0 second intervals. The suspension is then spun at 13,000 RPM in a table top Eppindorf centrifuge for 20 minutes. The supernatant is added to the culture at a concentration of between about 0.1 and 5.0 mg/ml. After about three months the dermal fibroblasts are seen to express the CD34 marker.

It is believed that human fetal, newborn and adult tissues contain subsets of stem cells that have the potential to differentiate or to be transdifferentiated into cells of many phenotypes. The invention comprises the use of skin fibroblastic cells from any of the three germlayers (mesoderm, endoderm and ectoderm) regardless of the age of the organism have subpopulations of multipotent cells useful for building replacement tissues. Excluded from the claims are patented proprietary methods of isolating and using bone marrow mesenchymal stem cells. The manufacture of specific signaling complexes by the methods referred to herein allow one to identify the multiplicity of phenotypes in which the fetal skin fibroblasts can differentiate. For example, fetal dermal fibroblasts have overturned the classical notion of the germ-layer barrier, by differentiating into pancreas and liver phenotypes, both of which are endodermal derivatives.

Signaling complexes in the forms of ADMAT, liquid extracts and fractions from developing animal tissues at different stages of development are prepared by the method described in U.S. Pat. No. 5,800,537, U.S. application Ser. No. 60/251,125 referred to herein, and a concurrently filed U.S. application referred to herein, the entire contents of which are herein incorporated by reference.

Some animal sources include fetal or newborn pigs, cows and sheep. Any and all developing tissues from animals, e.g., brain, liver, muscle, skin, heart, lung, bone, tendon, pancreas and kidney tissue, can provide signaling complexes.

Using tissue specific signaling complexes, experimental results have shown that mouse embryonic stem cells can be predictably induced into a variety of cell types, e.g., serum albumin producing liver cells, beating myocytes, or bone cells. Additionally, as described herein, tissue specific signaling complexes have been found to be responsible for inducing cultured human fetal skin fibroblasts to become many different cell types, e.g., bone cells, cartilage cells, insulin secreting cells, glucagon secreting cells and chymotrypsin secreting cells.

### EXAMPLE 1

### The Preparation of Human Cartilage from Human Fetal Skin Cells in Vitro

Fetal skin at 24 weeks of age is collected, cut into small pieces and treated with trypsin at room temperature for 30 min. The cells are resuspended in medium containing 10% FBS in DMEM. The cells in suspension are decanted with the supernatant and plated on to culture plates to establish a primary culture of the fibroblastic skin cells.

The cells are seeded into a collagen scaffold in three dimensions before the addition of cartilage-specific signaling complex. The complex is prepared by making extracellular microparticulates as described in U.S. Pat. No. 5,800,537 referred to herein, and extracting them with DMEM. The total extract is spun at 4000 RPM for 30 minutes at 4° C., passed through two layers of 1.0 mm pore size cheese cloth and then through a 0.8 μm syringe filter before adding 30 μg of signaling complex to 1 ml of culture medium now containing 0.5% FBS. In samples which receive the cartilage-specific signaling complex, cartilage forms in vitro in approximately three months. In controls which have not received the signaling complex, no cartilage forms.

EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. The use of fibroblastic cells as stem cells for cell differentiation, histiogenesis, organogenesis, cell therapy, tissue engineering, and tissue and organ regeneration comprising:
a. exposing fibroblastic cells to tissue specific signals to induce said cells to divide, engage in morphogenesis, differentiate into phenotypes unlike that of the dermal fibroblast, and or form tissues and or organs.

2. The method of claim 1, wherein said cells are derived from fetal or adult dermis and have the capacity to respond to said tissue specific signals.

3. The method of claim 1, wherein said tissues and or organs are unlike the dermis of the skin.

4. The method of claim 1, wherein said method further comprises creating large cultures of said cells of numbers between about 10² and 10¹⁵, which can be induced by said tissue specific signals to divide and differentiate along particular pathways of development for the purpose of forming tissues and or organs.

5. The method of claim 1, wherein said method further comprises delivering said cells to tissues of a recipient alone or in a delivery vehicle.

6. The method of claim 6, wherein said delivery comprises injecting said cells into said tissues of a recipient.

7. A tissue or organ-equivalent produced by the method of claim 1 comprising cells, signaling complexes and a scaffold.

8. The use of a tissue or organ-equivalent produced by the method of claim 1 in vitro or for implantation in vivo.

9. The method of claim 1, wherein said cells are cultivated for about three months in a medium comprising an extract derived from mES so that said cells express the stem cell marker CD34.
